(19) United States
(12) Patent Application Publication
(10) Pub. No.: US 2022/0325333 A1
(43) Pub. Date: Oct. 13, 2022

(54) PRIMER SET OF LAMP-LFD VISUAL DETECTION FOR DETECTING LEAF CURL VIRUS OF MELIA AZEDAEACHL AND DETECTION METHOD

(71) Applicant: CHINA JILIANG UNIVERSITY, (US)

(72) Inventors: Kai SUN, Hangzhou (CN); Huixin LU, Hangzhou (CN); Xin ZHOU, Hangzhou (CN); Xiaoping YU, Hangzhou (CN); Pengjun ZHANG, Hangzhou (CN); Chuanlin YIN, Hangzhou (CN)

(73) Assignee: CHINA JILIANG UNIVERSITY, Hangzhou, ZJ (CN)

(21) Appl. No.: 17/724,526

(22) Filed: Apr. 20, 2022

(30) Foreign Application Priority Data
Sep. 2, 2021 (CN) 202111026220.8

(51) Int. Cl. C12Q 1/6844; C12Q 1/70

(52) U.S. Cl. CPC C12Q 1/6844; C12Q 1/701

(57) ABSTRACT

The present invention discloses a primer set of LAMP-LFD detection for detecting a leaf curl virus of Melia azedaeach L. and a detection method, and belongs to the field of detection of crop diseases. The primer set of LAMP-LFD visual detection includes five primers: CLSV-F3, CLSV-B3, CLSV-FIP, CLSV-BIP and CLSV-LB and one probe primer: CLSV-Pb. The present invention also provides a visual detection method for a leaf curl virus of Melia azedaeach L., which is a combination of a loop-mediated isothermal amplification method and a lateral flow dipstick to establish an LAMP-LFD method to detect CLSV, so that the results of LAMP amplification detection are more obvious and intuitive. The present invention has the characteristics of high specificity, high sensitivity and simple operation, and is suitable for substrate and field use.

### TECHNICAL FIELD

The present invention relates to the technical field of detection, identification and control of crop diseases, in particular to a primer set of LAMP-LED visual detection for detecting a leaf curl virus of Melia azedaeach L. and a detection method.

### REFERENCE TO SEQUENCE LISTING

The substitute sequence listing is submitted as an ASCII formatted text filed via EFS-Web, with a file name of “Substitute_Sequence_Listing_SJDL-USP1220955.TXT”, a creation date of Jun. 26, 2022, and a size of 14,600 bytes. The substitute sequence Listing filed via EFS-Web a part of the specification and is incorporated in its entirety by reference herein.

### BACKGROUND

Melia azedaeach L. is a deciduous tree of Meliaceae. It originates in Australia and Southeast Asia, and is often distributed in Sichuan, Yunnan, Guizhou, Jiangsu and Zhejiang provinces in China. The plant grows rapidly on moist fertile soil and does not have strict soil requirements. The plant can grow in acidic soil, neutral soil and limestone areas, and is a good afforestation tree species in plains and low-altitude hilly areas. Melia azedaeach L. is also a medicinal plant. Its whole plant or specific parts (leaves, stems and roots) can be used as medicine. Melia azedaeach L. is often used to treat diseases of diarrhea, diabetes, rheumatism and hypertension. At present, the research of Melia azedaeach L. mainly focuses on cultivation and medicinal activity. The leaf curl of Melia azedaeach L. occurs generally, and shows an outbreak trend. Susceptible plants show symptoms of dwarf plants and curled leaves, and the control situation is not optimistic. If the diseases are allowed to spread further, the forestry in China will be greatly influenced.

The existing disease detection technologies include conventional PCR, nested PCR and real-time fluorescence quantitative PCR, but the conventional PCR, the nested PCR and the real-time fluorescence quantitative PCR have long detection time, complicated operation, time consumption and labor consumption. Loop-mediated. isothermal amplification (LAMP) is a nucleic acid amplification method different from the principle of PCR. Without complicated reaction procedures and highly technical instruments, it only requires a reaction in an ordinary PCR instrument at 60° C-65° C. for 30-60 min under the action of strand displacement DNA polymerase to obtain 109 amplification of nucleic acid. The advantages are high sensitivity, strong specificity, short reaction time and simple operation, and the results can be observed with the naked eyes by adding fluorescent dyes to the product. The disadvantages are that the primer design is complicated, and an aerosol phenomenon is extremely easy to occur, resulting in false positive. Therefore, for the accurate interpretation of the product, a lateral flow dipstick (LFD) method is needed. The LED is a combination of immunochromatography and molecular biology means to anchor specific fluorescein antibodies on the LED to react with the fluorescein labeled on the LAMP product, so that the sample forms a colored detection line on the LFD, thereby detecting a correct target product and greatly reducing the probability of the false positive.

According to the above description, the present invention provides a pruner set of LAMP-LFD visual detection for a leaf curl virus of Melia azedaeach L. and a detection method. At present, there is no report in China and abroad that the technology is applied to the leaf curl virus of Melia azedaeach L.

### SUMMARY

The problem to be solved by the present invention is the lack of a detection means for a leaf curl virus of Melia azedaeach L. at present. Therefore, the present invention provides a primer set of LAMP-LFD detection for a leaf curl virus of Melia azedaeach L. and a detection method, with high detection speed, low cost, high sensitivity and high accuracy.

Technical solutions adopted to solve the above problems by the present invention are as follows:

The present invention firstly provides a primer set of LAMP-LED visual detection for detecting a leaf curl virus of Melia azedaeach L., comprising five primers: CLSV-F3, CLSV-B3, CLSV-FIP, CLSV-BIP and CLSV-LB and one probe primer: CLSV-FITC BIP, with primer sequences as follows:

CLSV-F3:
(SEQ ID NO: 1)
5′-GAGAAATGGAGAAACAAACGT-3′;
CLSV-B3:
(SEQ ID NO: 2)
5′-CAACCTTTCCAAATCTCTGT-3′;
CLSV-FIP:
(SEQ ID NO: 3)
5′-GTCCCTTCTCTTCCCTCAGCGAGAATGATGTATCCCACGG-3′;
CLSV-BIP:
(SEQ ID NO: 4)
5′-CTGTCCCAGATAAGAAGAGTGTTCCCTGATCCTGAATATGTGTTGT-
3′;
CLSV-LB:
(SEQ ID NO: 5)
5′-TCGCTAGAGGATCCTGCTAC-3′;
probe primer CLSV-FITC PB:
(SEQ ID NO: 6)
5′-GGACAGAATATTCTGTGTC-3′;

wherein the 5′ end of CLSV-FIP is labeled with biotin; the 5′ end of probe CLSV-FITC Pb is labeled with fluorescein isothiocyanate.

The present invention also provides an LAMP-LTD visual detection method for a leaf curl virus of Melia azedaeach L. using the above primer set, including the following steps:

1) extracting DNA of virus-infected tissue;

2) loop-mediated isothermal amplification reaction: making the obtained DNA conduct a loop-mediated isothermal amplification reaction in an amplification reaction system which comprises 2.5 μL of 10×Buffer, 10 mmol/L of MgSO₄, 1.4 mmol/L of dNTP Mix, 1.6 μmol/L of CLSV-FIP, 1.6 μmol/L of CLSV-BIP, 0.2 μmol/L of CLSV-F3, 0.2 μmol/L of CLSV-B3, 0.4 μmol/L of CLSV-LB, 0.5 μL of 160U Bst 2.0 DNA polymerase, 1 μL of template and sterile double distilled water added to make up to 25 μL.;

3) LFD detection: replacing the CLSV-BIP in the above amplification reaction system with an equimolar amount of fluorescein isothiocyanate-labeled probe primer CLSV-FITC BIP; reacting at 60° C. for 1 h; after the reaction is ended, adding 100 μL of HybriDetect Assay Buffer and mixing uniformly; vertically inserting the detection end of a lateral flow dipstick into fluid to be tested; standing for 5 min; and observing the results with naked eyes.

Preferably, in the LAMP-LFD visual detection method for the leaf curl virus of Melia azedaeach L., in an optimal LAMP reaction system, the concentration of MgSO₄ is 10 mmol/L, and the concentration of dNTP Mix is 1.4 mmol/L.

Preferably, in the LAMP-LTD visual detection method for the leaf curl virus of Melia azedaeach L., the reaction temperature of LAMP is 60° C.

Preferably, in the LAMP-LFD visual detection method for the leaf curl virus of Melia azedaeach L., the reaction time of LAMP is 60 min.

The present invention also provides an application of the primer set of LAMP-LFD visual detection for detecting the leaf curl virus of Melia azedaeach L. in diagnosis, detection and identification of the leaf curl virus of Melia azedaeach L.

Compared with the prior art through the above experimental solution, the present invention provides a primer set for LAMP-LFD-based visual detection and a detection method. The main advantage is that the FITC primer set and the detection method can be used for the detection of plants infected by the leaf curl virus of Melia azedaeach L. in production practice rapidly, sensitively and efficiently, can also be used for early diagnosis, monitoring and identification of the leaf curl virus of Melia azedaeach L., and provides reliable technical support and theoretical basis for prevention and control of the diseases caused by the leaf curl virus of Melia azedaeach L. Compared with the prior art, the present invention has the advantages that:

(1) Strong specificity: the present invention designs primers and probes required for LAMP-LFD according to the gp3 gene of the leaf curl of Melia azedaeach L.

(2) High sensitivity: the detection sensitivity of LAMP-LFD to the leaf curl virus of Melia azedaeach L. can reach 1pg at DNA level, which is 1000 times higher than that of conventional PCR detection.

(3) Convenience and quickness: the amplification time of the present invention is short, which only takes 60 min. The reaction procedure is simple and can be kept at 60° C. The LFD detection is accurate and fast, without the need for large complex instruments, and one person can complete all experimental steps within 1.5 h.

(4) To sum up, the present invention has high accuracy, sensitivity and convenience and simple operation for the detection of the leaf curl virus of Melia azedaeach L., and can satisfy the needs of field detection.

### DESCRIPTION OF DRAWINGS

FIG. 1 shows electrophoresis results after LAMP amplification for a leaf curl virus of Melia azedaeach L. under different MgSO₄ concentrations.

FIG. 2 shows electrophoresis results after LAMP amplification for a leaf curl virus of Melia azedaeach L. under different dNTP concentrations.

FIG. 3 shows electrophoresis results after LAMP amplification for a leaf curl virus of Melia azedaeach L. at different temperatures.

FIG. 4 shows electrophoresis results after LAMP amplification for a leaf curl virus of Melia azedaeach L. at different reaction times.

FIG. 5 shows electrophoresis results after an ordinary PCR reaction under templates with different concentrations.

FIG. 6 shows electrophoresis results after LAMP amplification under templates with different concentrations.

FIG. 7 shows LFD detection results under templates with different concentrations,

FIG. 8 shows results after an ordinary PCR reaction of a blind sample.

FIG. 9 shows LAMP-LFD detection results of a blind sample.

### DETAILED DESCRIPTION

The present invention will be further illustrated and described below in combination with the drawings and the embodiments. However, the described embodiments are not all of the embodiments of the present invention. Based on the embodiments in the present invention, all other embodiments without substantial change made by those skilled in the art based on the present invention belong to the protection scope of the present invention.

Embodiments of the present invention disclose a primer set of LAMP-LED visual detection for detecting a leaf curl virus of Melia azedaeach L. and a detection method. Raw materials required in the embodiments are all purchased from commercially available channels.

### EMBODIMENT 1

Design for the primer set of LAMP-LED detection for the leaf curl virus of Melia azedaeach L.

1. Extraction of genome DNA of the leaf curl virus of Melia azedaeach L.:

(1) A CTAB method is used to extract the genome DNA of the leaf curl virus of Melia azedaeach L., including the following specific steps: heating a CTAB extracting solution (2×) in a 65° C. water bath for 30 min in advance, grinding freeze-dried samples into powder with liquid nitrogen, transferring the powder into a 1.5 mL centrifuge tube, adding 8000 of preheated CTAB, mixing uniformly, and keeping at 65° C. for 30 min-60 min, centrifuging at 12000 r/min for 10 min at room temperature, and discarding the precipitates; then, adding the same volume of phenol: chloroform: isoamyl alcohol (25:24:1) as the supernatant after centrifugation, mixing uniformly, placing in a fuming cupboard for 15 min, and then centrifuging at 12000 r/min for 10 min at room temperature (a layering phenomenon exists in the tube, the supernatant has nucleic acid, the middle is a protein layer, and the bottom has impurities); carefully pipetting the supernatant into a new 1.5 mL centrifuge tube (this step can be repeated until the protein layer is not visible to the naked eyes); adding ⅔ volume or equal volume of pre-cooled isopropanol, gently inverting up and down to mix uniformly, standing for 5-10 min, and centrifuging at 4° C. and 10000/12000 r/min for 10 min in a centrifugal machine; removing the supernatant, adding 1 mL of 75% ethanol, washing the DNA gently by inversion, centrifuging at 4° C. and 12000 r/min for 10 min (repeating this step once), and discarding the supernatant; opening a cap, naturally drying the residual ethanol on the wall of the tube, adding 200 μl of sterile double distilled water, dissolving the DNA, and taking a water bath at 50° C. for 2.0 min (to remove volatiles); adding 1 μl of 10 mg/mL RNase, keeping at 37° C. for 60 min (removing the RNA in this step), and then storing at −20° C.

(2) PCR reaction system: 25 μl of reaction system, including 12.5 μl of KOD ONE enzyme, 1 μl of each primer, 1 μl of template DNA extracted in step (1), and double distilled water filled to 25 μl.

(3) PCR reaction system: the reaction system in step (2) is put under the following reaction conditions: pre-denaturation at 98° C. for 1 min; denaturation at 98° C. for 15 s, annealing at 57° C. for 15 s, and extension at 68° C. for 1 min, with 33 cycles; re-extension at 68° C. for 5 min; holding at 12° C.

(4) The reaction product obtained in step (3) is delivered to Hangzhou Youkang Biotechnology Co., Ltd. for sequencing to obtain the nucleotide sequences of the leaf curl virus gp3 of Melia azedaeach L.

2. Primer design:

LAMP primers are designed according to gp3 primer sequences. The primer sequences are as follows:

CLSV-F3:
(SEQ ID NO: 1)
5′-GAGAAATGGAGAAACAAACGT-3′;
CLSV-B3:
(SEQ ID NO: 2)
5′-CAACCTTTCCAAATCTCTGT-3′;
CLSV-FIP:
(SEQ ID NO: 3)
5′-GTCCCTTCTCTTCCCTCAGCGAGAATGATGTATCCCACGG-3′;
CLSV-BIP:
(SEQ ID NO: 4)
5′-CTGTCCCAGATAAGAAGAGTGTTCCCTGATCCTGAATATGTGTTG
T-3′;
CLSV-LB:
(SEQ ID NO: 5)
5′-TCGCTAGAGGATCCTGCTAC-3′;
probe CLSV-FIT-pb:
(SEQ ID NO: 6)
5′-GGACAGAATATTCTGTGTC-3′;

wherein the 5′ end of CLSV-FIP is labeled with biotin; and the 5′ end of probe CLSV-FITC-pb is labeled with fluorescein isothiocyanate.

### EMBODIMENT 2

Optimization for reaction conditions of LAMP in the present invention

1. Optimization for LAMP reaction system:

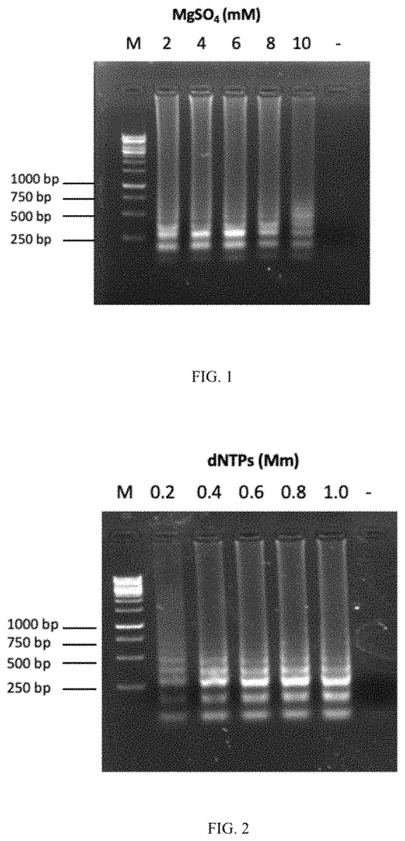

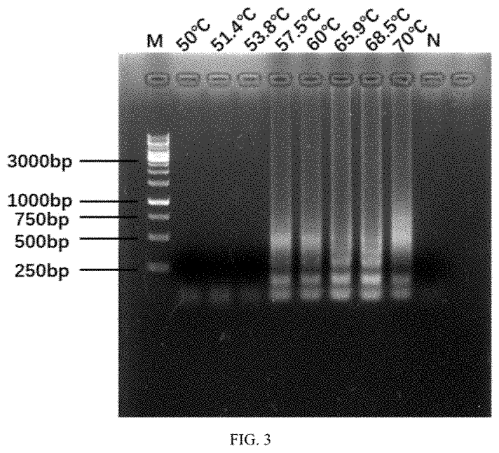

The LAMP reaction system is optimized repeatedly by using the primer set of the present invention, and finally the optimal concentrations of components in the reaction system are determined to be 10 mmol/L of MgSO₄, 1.4 mmol/L of dNTP Mix, 1.6 μmol of inner primer CLSV-FIP, 1.6 μmol of inner primer CLSV-BIP, 0.2 μmol/L of outer primer CLSV-F3, 0.2 μmol/L of outer primer CLSV-B3, 0.4 μmol/L of loop primer CLSV-LB, and 0.5 μL of 8000U BST 2.0 DNA polymerase. The optimization results of MgSO₄ and dNTP concentrations are shown in FIG. 1 and FIG. 2.

2. Optimization for LAMP reaction temperature:

According to the optimized system, temperature gradients of 50° C.-70° C. are set on a PCR instrument (gradients are: 50° C., 51.4° C., 53.8° C., 57.5° C., 60° C., 65.9° C., 68.5° C. and 70° C. respectively). Blank control is set. The amplified products are subjected to agarose gel electrophoresis, and electrophoretograms after amplification of the LAMP reaction system for the leaf curl virus of Melia azedaeach L. at different temperatures are shown in FIG. 3. Experimental results show that the reaction cannot be carried out at 57° C. Within 57° C-70° C., the amplification efficiency is gradually increased as the temperature rises; and the amplification efficiency is not changed much after 60° C. Therefore, the optimal reaction temperature is selected as 60° C. in the present invention.

3. Optimization for LAMP reaction time:

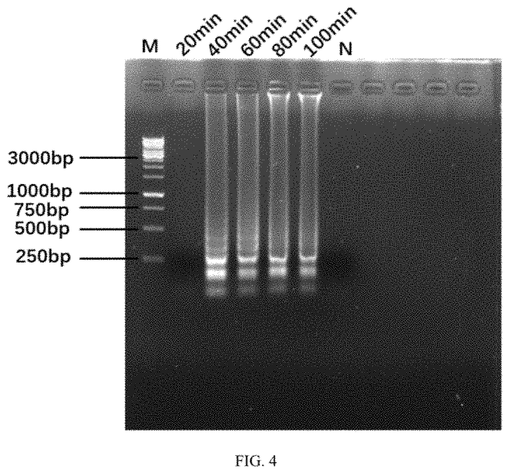

According to the optimized system, the reaction times are 20 min, 40 min, 60 min, 80 min and 100 min. The blank control is set. LAMP products with different reaction times are subjected to agarose gel electrophoresis. Electrophoretograms after amplification of the LAMP reaction system for the leaf curl virus of Melia azedaeach L. at different time are shown in FIG. 4. The experimental results show that products arc outputted after 40 min. In order to increase the yield, the optimal reaction time is selected as 60 min in the present invention.

4. Determination of LAMP reaction sensitivity:

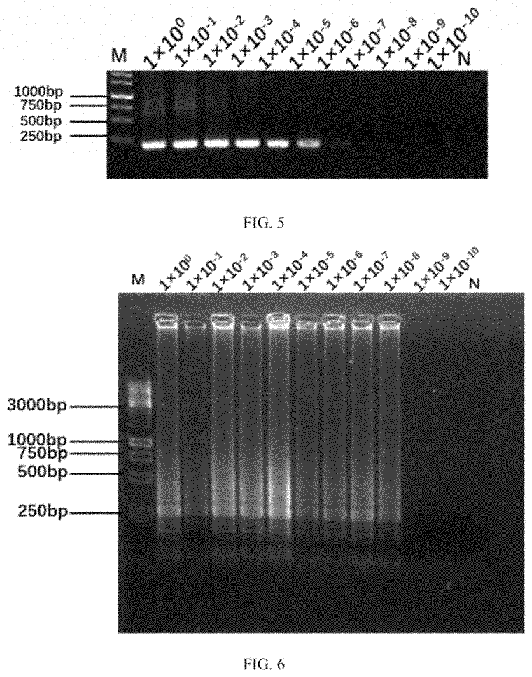

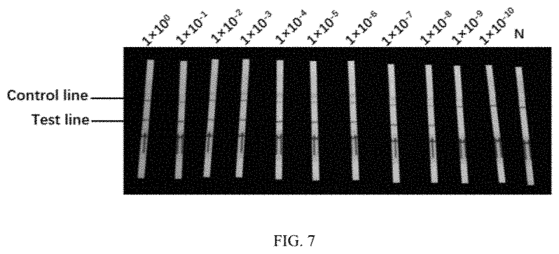

The DNA of the leaf curl virus of Melia azedaeach L. is extracted according to the method of step 1 in embodiment 1, and diluted by 10-fold gradient. The DNA of the leaf curl virus of Melia azedaeach L. at an original concentration of 10ng/μl is selected for dilution. Negative control is set with 10⁰, 10⁻¹, 10⁻², 10⁻³, 10⁻⁴, 10⁻⁵, 10⁻⁶10⁻⁷, 10⁻⁸, 10⁻⁹and 10⁻¹⁰ as templates. An ordinary PCR reaction is conducted firstly. Electrophoretograms are shown in FIG. 5, and amplification is conducted according to the system optimized in (1). Electrophoretograms after amplification of the LAMP reaction system for the leaf curl virus of Melia azedaeach L. at different template concentrations are shown in FIG. 6. Two experimental results are compared. It is obvious that LAMP is more sensitive than ordinary PCR, and is decreased as the concentration is decreased. The gradient dilution samples are subjected to LFD detection, as shown in FIG. 7. It is obvious that the detection amount is gradually decreased as the concentration is decreased. The LAMP sensitivity of the leaf curl virus of Melia azedaeach L. is detected as 10⁻⁸ times of the original concentration by using the primer set provided by the present invention.

### EMBODIMENT 3

The leaf curl virus of Melia azedaeach L. is detected in plant blind samples by using the LAMP-LFD technology of the present invention.

Nine unknown samples are taken and CLSV-specific primers are used.

CLSV-d1-F:
GAGAATGATGTATCCCACGG;
CLSV-d1-R:
CCTGATCCTGAATATGTGTTGT

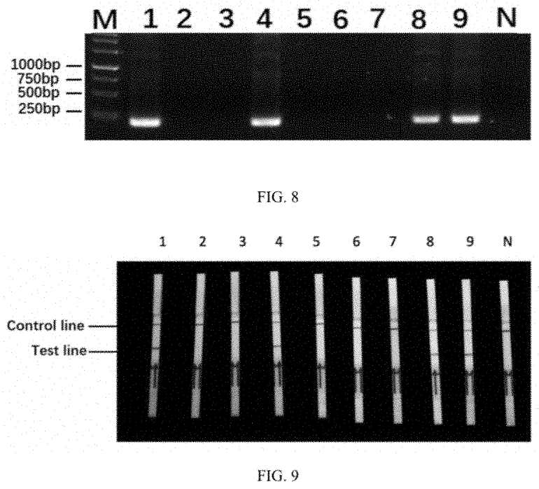

PCR amplification is conducted on the samples, and the size of a target band is 143 bp. In order to conduct agarose gel electrophoresis on the amplification results, the blank control is set. The results are shown in FIG. 8, wherein 1#, 4#, 8# and 9# are virus positive samples. The nine unknown samples are subjected to the LAMP-LFD visual detection of the present invention, and the blank control is set. The results are shown in FIG. 9. Similarly, 1#, 4#, 8# and 9# are obtained as positive results. Experiments show that the primer set of the present invention has high accuracy for the leaf curl virus of Melia azedaeach L.

What is claimed is:

1. A primer set of LAMP-LFD visual detection for detecting a leaf curl virus of Melia azedaeach L., comprising five primers: CLSV-F3, CLSV-B3, CLSV-FIP, CLSV-BIP and CLSV-LB and one probe primer: CLSV-FITC Pb, with primer sequences as follows:
CLSV-F3:
5′-GAGAAATGGAGAAACAAACGT-3′;
CLSV-B3:
5′-CAACCTTTCCAAATCTCTGT-3′;
CLSV-FIP:
5′-GTCCCTTCTCTTCCCTCAGCGAGAATGATGTATCCCACGG-3′;
CLSV-BIP:
5′-CTGTCCCAGATAAGAAGAGTGTTCCCTGATCCTGAATATGTGTTG
T-3′;
CLSV-LB:
5′-TCGCTAGAGGATCCTGCTAC-3′;
probe primer CLSV-FITC Pb:
5′-GGACAGAATATTCTGTGTC-3′;
wherein the 5′ end of CLSV-FIP is labeled with biotin; the 5′ end of probe CLSV-FITC Pb is labeled with fluorescein isothiocyanate.

2. An LAMP-LFD visual detection method for a leaf curl virus of Melia azedaeach L. using the primer set of claim 1, comprising the following steps:
S1. extracting DNA of virus-infected tissue;
S2. loop-mediated isothermal amplification reaction: making the obtained DNA conduct a loop-mediated isothermal amplification reaction in an amplification reaction system which comprises 2.5 μL of 10×Buffer, 5-15 mmol/L of MgSO₄, 0.5-1.0 mmol/L of dNTP Mix, 1.6 μmol/L of CLSV-FIP, 1.6 μmol/L of CLSV-BIP, 0.2 μmol/L of CLSV-F3, 0.2 mol/L of CLSV-B3, 0.4 μmol/L of CLSV-LB, 0.5 μL of 160U Bst 2.0 DNA polymerase, 1 μL of template and sterile double distilled water added to make up to 25 μL;
S3. LFD detection: replacing the CLSV-BIP in the above amplification reaction system with an equimolar amount of fluorescein isothiocyanate-labeled probe primer CLSV-FITC Pb; reacting at 60-65° C. for 40 min-60 min; after the reaction is ended, adding 100 μL of HybriDetect Assay Buffer and mixing uniformly; vertically inserting the detection end of a lateral flow dipstick into fluid to be tested; standing for 5 min; and observing the results with naked eyes.

3. The LAMP-LFD visual detection method for the leaf curl virus of Melia azedaeach L. according to claim 2, wherein in an optimal LAMP reaction system, the concentration of MgSO₄ is 10 Mm, and the concentration of dNTP Mix is 0.6 Mm.

4. The LAMP-LFD visual detection method for the leaf curl virus of Melia azedaeach L. according to claim 2, wherein the reaction temperature of LAMP is 60° C.

5. The LAMP-LFD visual detection method for the leaf curl virus of Melia azedaeach L. according to claim 2, wherein the reaction time of LAMP is 60 min.

6. An application of the primer set of LAMP-LFD visual detection for detecting the leaf curl virus of Melia azedaeach L. of claim 1 in diagnosis, detection and identification of the leaf curl virus of Melia azedaeach L.


SEQUENCE LISTING (6 sequences)

SEQ ID NO: 1 - DNA, 21 bp, Artificial Sequence
  Synthetic primer
gagaaatggagaaacaaacgt

SEQ ID NO: 2 - DNA, 20 bp, Artificial Sequence
  Synthetic primer
caacctttccaaatctctgt

SEQ ID NO: 3 - DNA, 40 bp, Artificial Sequence
  Synthetic primer
gtcccttctcttccctcagcgagaatgatgtatcccacgg

SEQ ID NO: 4 - DNA, 46 bp, Artificial Sequence
  Synthetic primer
ctgtcccagataagaagagtgttccctgatcctgaatatgtgttgt

SEQ ID NO: 5 - DNA, 20 bp, Artificial Sequence
  Synthetic primer
tcgctagaggatcctgctac

SEQ ID NO: 6 - DNA, 19 bp, Artificial Sequence
  Synthetic primer
ggacagaatattctgtgtc